(19) United States
(12) Patent Application Publication
(10) Pub. No.: US 2010/0152673 A1
(43) Pub. Date: Jun. 17, 2010

(54) Syringe

(76) Inventor: Chen-Yi FANG, Taichung City (TW)

Correspondence Address: Dr. BANGER SHIA;Patent Office of Bang Shia, 102 Lindencrest Ct, Sugar Land, TX, 77479-5201, US

(21) Appl. No.: 12/337,536

(22) Filed: Dec. 17, 2008

(51) Int. Cl. A61M 5/315

(52) U.S. Cl. 604/218

(57) ABSTRACT

A syringe includes a sleeve fitted in a receiving hole of the front end of a syringe cylinder. A limiting element is axially extended from an inner periphery of a joint portion of a combining section and a tapered section of the sleeve. The limiting element includes a plurality of spaced roots from which larger-diameter circular projections, by which an opening is defined, are extendedly secured. The diameter of the opening is narrower than the intermediate and bottom section of a tenon. Therefore, a larger deformation space of the limiting element is obtained for the smooth insertion of a push rod into the sleeve.

### BACKGROUND OF THE INVENTION

1. Field of the Invention

The present invention relates to a syringe, and more particularly to a safety syringe that allows a push rod to insert into a sleeve thereof smoothly.

2. Description of the Prior Art

Syringe is an indispensable auxiliary instrument in medical treatment, for example, AU serial No. 2006100012 entitled safety syringe discloses it can effectively reduce operational injury due to carelessness, however such a conventional syringe still has some following defects that could be improved:

As depicted in AU Serial No. 2006100012, a tapered hole surrounded by a plurality of equally wide buckles is provided in a sleeve, hence a tenon cannot be easily inserted into the tapered hole, due to the buckles are arranged in a equally wide manner.

The present invention has arisen to mitigate and/or obviate the afore-described disadvantages.

### SUMMARY OF THE INVENTION

The primary objective of the present invention is to provide a syringe that can obtain a larger deformation space for the smooth insertion of a push rod into a sleeve thereof.

To achieve the above object, a limiting element is axially extended from an inner periphery of a joint portion of the combining section and the tapered section of the syringe of the present invention. The limiting element is defined with a plurality of spaced roots from which larger-diametered circular projections are extended respectively. An opening is defined by the circular projections and a diameter thereof is narrower than intermediate and bottom sections of the tenon, such that as the tenon of the push rod is inserted into the opening, by using the elasticity of the roots, the tenon can smoothly passing across and engaging in the limiting element.

The present invention will become more obvious from the following description when taken in connection with the accompanying drawings, which show, for purpose of illustrations only, the preferred embodiments in accordance with the present invention.

### BRIEF DESCRIPTION OF THE DRAWINGS

FIG. 1 is an exploded perspective view of the present invention;

FIG. 2 is a schematic cross sectional view of the present invention;

FIG. 2A is a schematic cross sectional view illustrating the tapered section of the present invention;

FIG. 2B is a partial enlarged cross sectional view of the FIG. 2;

FIG. 3 shows the schematic cross sectional view about the application of the present invention.

### DETAILED DESCRIPTION OF THE PREFERRED EMBODIMENTS

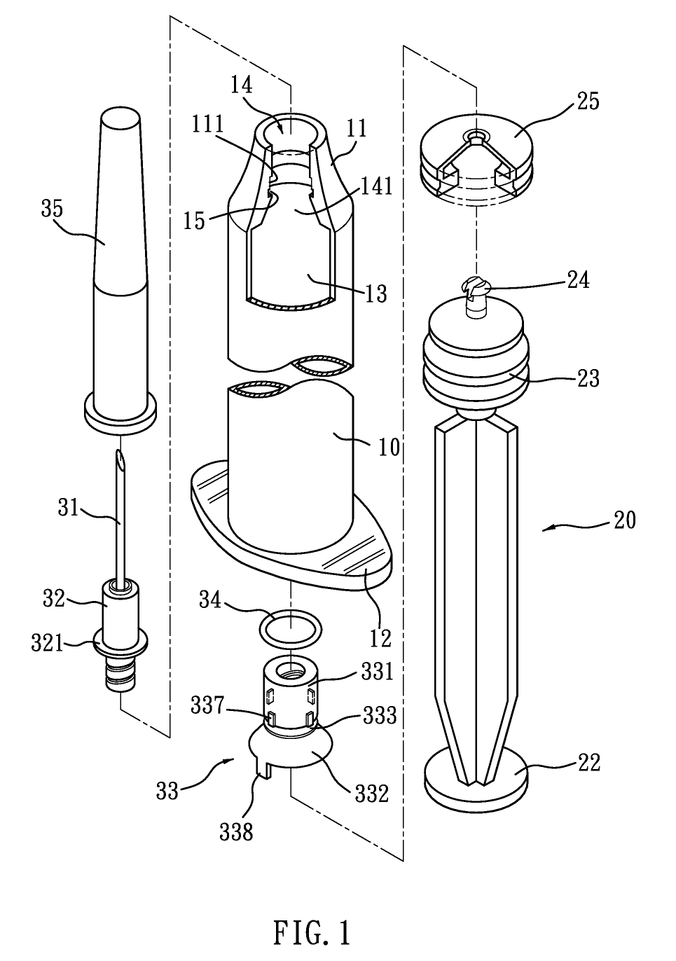

Referring to FIG. 1, the syringe of the present invention is illustrated. The present invention has the following elements.

A syringe cylinder 10 is a hollow cylinder. One end of the syringe cylinder 10 is formed with a reduced section 11 and another end thereof is an enlarged flange 12. An interior of the syringe cylinder 10 is formed with a receiving chamber 13. An interior of the reduced section 11 is formed with a receiving hole 14 which has a reduced hole diameter and is communicated to the receiving chamber 13. A diameter of the receiving chamber 13 is slightly larger than that of the receiving hole 14. A joint portion of the receiving chamber 13 and the receiving hole 14 is formed with a stop edge. The receiving hole 14 is formed with a trumpet-shaped opening 141 which has a smaller end at an outer side and has larger end at a side connected to the receiving chamber 13. An inner wall of the reduced section 11 is formed with an annular recess 15. More specifically, the annular recess 15 is disposed on the inner wall of a joint portion of the receiving hole 14 and the trumpet-shaped opening 141.

A push rod 20 is movably inserted in the receiving chamber 13 of the syringe cylinder 10 and is movable linearly in the receiving chamber 13. The push rod 20 has an enlarged distal end which is formed as an annular ring 22. A front end of the push rod 20 is formed with a combining portion 23 which is formed by a plurality of spaced disk. A front end of the combining portion 23 extends with a tenon 24. An elastic plug 25 encloses the tenon 24 and then is installed to the combining portion 23.

A needle set having a syringe needle 31, a seat 32 and a rubber sleeve 33. The syringe needle 31 is inserted and fixed to the seat 32. An outer expanded round buckling ring 321 is extended from the seat 32. A cover 35 resists against the seat 32 so as to cover upon the syringe needle 31. A lower side of the buckling ring 321 of the seat 32 is formed with an outer thread for locking the sleeve 33.

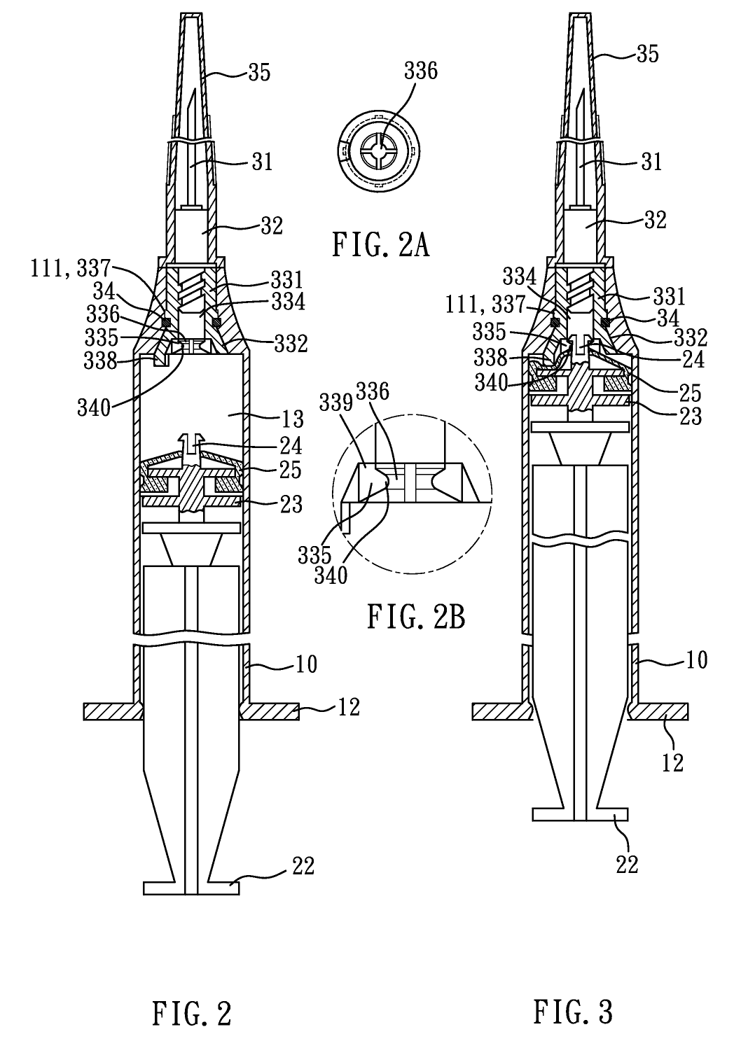

Referring to FIGS. 2 and 3, the sleeve 33 is received in the receiving hole 14. To tightly seal the sleeve 33 in the receiving hole 14, the sleeve 33 is formed as a trumpet to match the shape of the receiving hole 14. The sleeve 33 includes a round combining section 331, a tapered section 332 and a tunnel 334 axially passing therethrough. An outer periphery of the joint portion of the combining section 331 and the tapered section 332 is formed with an annular slot 333. A rubber O ring 34 is sleeved into the annular slot 333. The O ring 34 is also located at the annular recess 15 so that the sleeve 33 is tightly engaged to the reduced section 11 and is not separated from the injection direction. Besides, the maximum diameter of the tapered section 332 is smaller than that of the receiving chamber 13. Furthermore, an inner thread at an inner wall of the combining section 331 mates with the outer thread of the seat 32 to secure both. A bottom of the tapered section 332 flushes with the trumpet-shaped opening 131 as the sleeve 33 is fitted into the receiving hole 14. From the bottom of the tapered section 332 axially extending a protrusion 338 toward the receiving chamber 13 direction. Normally, the protrusion 338 protrudes into the receiving chamber 13. When the push rod 20 is completely pushed into the receiving chamber 13, the elastic plug 25 at the front end of the push rod elastically resists against the protrusion 338.

Now please refer to FIGS. 2A and 2B simultaneously. From an inner periphery of a joint portion of the combining section 331 and the tapered section 332 is axially extended a limiting element 335. The limiting element 335 is defined with a plurality of spaced roots 339 from which larger-diametered circular projections 340 are extended respectively. One face of each root 339 flushes an inner periphery of the combining section 331, and the circular projections 340 extends toward an axis of the tenon 24. An opening 336 is defined by the circular projections 340, and a diameter thereof is narrower than intermediate and bottom sections of the tenon 24. In addition, at a predetermined position of an outer periphery of the combining section 331 is spaced mounted four tabs 337, and on the inner wall of the reduced section 11 is partially disposed four locking grooves 111 corresponding to the tabs 337.

Because the diameter of each root is smaller than that of each circular projection, the limiting element 335 can obtain a lager deformation space. Therefore, when the tenon 24 of the push rod 20 is inserted from the opening 336, the limiting element 335 stretches outward with the thrush force to widen the opening 336, such that the push rod 20 can smoothly pass across the limiting element 335 for a smooth injection of medicament as well as preventing the tenon 24 from loosen out of the circular projections 340.

It is to be noted that on the combining section 331 of the sleeve 33 is secured the spaced retaining tabs 337 which are limited in the locking grooves 111 of the inner wall of the reduced section 11, such that during the process that the seat 32 and the sleeve 33 are screwed together, the sleeve 33 is free from the trouble that the sleeve 33 rotates along with the seat 32.

The invention is not limited to the above embodiment but various modifications thereof may be made. It will be understood by those skilled in the art that various changes in form and detail may made without departing from the scope and spirit of the present invention.

What is claimed is:

1. A syringe comprising:
a syringe cylinder being a hollow cylinder; one end of the syringe cylinder being formed with an reduced section and another end thereof being an enlarged flange; an interior of the syringe cylinder being formed with a receiving chamber; an interior of the reduced section being formed with a receiving hole communicated to the receiving chamber; the receiving hole being formed with a trumpet-shaped opening which has a smaller end at an outer side thereof and has a larger end at a side connected to the receiving chamber;
a push rod linearly movably inserted in the receiving chamber of the syringe cylinder; an elastic plug being installed at one end of the push rod, and an annular ring being formed at another end thereof; the elastic plug being slidable against an inner wall of the receiving chamber of the syringe cylinder; one side of the elastic plug being installed with a tenon;
a needle assembly comprising a syringe needle and a sleeve which is received in the receiving hole; the sleeve having a combining section, a tapered section and a tunnel axially passing therethrough; at a top of the combining section being provided with the syringe needle; a largest radius of the tapered section being smaller than that of the receiving chamber; the tapered section flushing with the trumpet-shaped opening as the sleeve being fitted into the receiving hole; wherein
from an inner periphery of a joint portion of the combining section and the tapered section is axially extended a limiting element; the limiting element is defined with a plurality of spaced roots from which larger-diametered circular projections are extended respectively; an opening is defined by the circular projections, and a diameter thereof is narrower than intermediate and bottom sections of the tenon, thereby as the tenon of the push rod is inserted from the opening, by using elasticity of the roots, the tenon is capable of smoothly passing across and engaging in the limiting element.

2. The syringe as claimed in claim 1, wherein at an inner wall of the reduced section is annularly disposed a annular recess, in an outer periphery of the joint portion of the combining section and the tapered section is provided with a small-diametered annular slot in which a O ring is fixed for engaging with the annular recess.

3. The syringe as claimed in claim 1, wherein the sleeve is made of rubber.

4. The syringe as claimed in claim 1, wherein at a predetermined position of an outer periphery of the sleeve is extendedly mounted at least one tab, and on the inner wall of said reduced section is partially disposed at least one locking groove corresponding to the tab.
